(19) United States
(12) Patent Application Publication
(10) Pub. No.: US 2009/0156569 A1
(43) Pub. Date: Jun. 18, 2009

(54) ORAL CONTRACEPTIVE SPRAY

(75) Inventor: Kees van der Voort Maarschalk, Oss (NL)

Correspondence Address: ORGANON USA, INC.;c/o Schering-Plough Corporation, 2000 Galloping Hill Road, Mail Stop: K-6-1, 1990, Kenilworth, NJ, 07033, US

(73) Assignee: N.V. Organon, Oss (NL)

(21) Appl. No.: 11/955,870

(22) Filed: Dec. 13, 2007

(51) Int. Cl. A61K 31/56; A61P 15/18

(52) U.S. Cl. 514/178

(57) ABSTRACT

The subject invention provides a liquid contraceptive formulation for oral transmucosal administration. The formulation comprises etonogestrel, optionally together with ethinyl estradiol but does not contain a matrix former or a percutaneous absorption promoter, which is a hydroxy acid or a salt thereof.

### RELATED APPLICATIONS

This application claims priority to the U.S. Provisional Application No. 60/874,308, entitled “ORAL CONTRACEPTIVE SPRAY,” filed Dec. 12, 2006, which is incorporated by reference in its entirety.

### BACKGROUND

The present invention relates to the field of female reproductive medicine, and in particular to a new form of human female contraception, namely an oral transmucosal contraceptive.

There is a constant need for methods for the safe, effective and convenient administration of hormonal contraceptive agents. Oral contraception with ingested dosage forms is commonly used but is associated with gastrointestinal tract absorption and drug first pass metabolism in the liver and/or gastrointestinal tract. Oral contraception with ingested dosage forms is therefore less efficacious when a patient is suffering from diarrhea or vomiting.

The oral mucosal route of administration is described in the literature: Tmax of the plasma versus time curve is usually shorter, Cmax is higher and AUC is higher when similar dosages as given via the oral mucosal route are compared with the oral ingestion route. The difference in AUC is usually attributed to prevention of a first pass effect and for that reason the oral mucosal route provides opportunities to increase exposure as expressed by a higher AUC and/or Cmax. The short Tmax and higher Cmax are usually also attributed to the immediate start of absorption of the active moiety. However, complete absorption via the oral mucosal membranes cannot be expected a priori, even at low doses: Temple et al. (1978), Archiv der Pharmazie, 311(6); 485-491 show that although absorption through the oral mucosal membranes is initially fast, the absorption by these tissues is also incomplete because the amount absorbed versus the time profile levels off at levels well below 100% absorbed. Moreover, it has been reported that the clearance of a compound from the mouth is fast as a result of e.g. swallowing (C. G. Wilson et al. (1987) Int. J. Pharm. 40; 119-123). This implies that for compounds with a low first pass metabolism, a large increase in exposure can not be expected a priori.

The literature describes transdermal and transmucosal administration: AU687013 (equivalent to EP655916) describes a contraceptive composition for transdermal administration comprising etonogestrel and describes that a typical transdermal spray contains a matrix former. US2004/0110732 describes a pharmaceutical composition for transmucosal administration comprising a progestin, an estrogen and at least one percutaneous absorption promoter which is a hydroxy acid or salt thereof. EP910339 describes a buccal aerosol pump spray which may contain a steroid compound. U.S. Pat. No. 6,506,742 describes an oral liquid formulation with norgestimate (for ingestion) useful as a liquid reference.

Hunt et al. (1998), Clin. Drug Invest. 15(6), 507-514 found that there was no difference in the extent of exposure of 3-ketodesogestrel, and hence in absorption and metabolism, from desogestrel tablets when compared with the reference solution which was orally ingested.

Etonogestrel (also named 3-ketodesogestrel, also named 13-ethyl-11methylene-17β-hydroxy-18,19-dinor-17α-pregnen-3-one) is a progestogen which is used in the form of its prodrug desogestrel in a variety of solid female contraceptive products. For example, Cerazette® is a progestogen-only solid oral contraceptive product comprising desogestrel. Another example is Marvelon® which is a solid oral contraceptive product comprising desogestrel in combination with ethinyl estradiol. Timmer and Mulders (2000), Clin. Pharmacokinet. 39(3): 233-242 show that exposure of etonogestrel after oral ingestion as its prodrug desogestrel has an oral bioavailability of about 80%, implying that absorption of desogestrel by the GI-tract as well as conversion from desogestrel to etonogestrel by first pass metabolism in the liver are almost complete.

It has now surprisingly been found that in fact there is still a great difference in the extent of exposure (as expressed by AUC and Cmax) to 3-keto desogestrel from tablets when compared with a solution for oral mucosal delivery. This is in spite of the expected incomplete absorption via the oral mucosal membranes and low first pass metabolism after oral ingestion. Etonogestrel (or its prodrug desogestrel or 3-β-hydroxy-desogestrel) optionally in combination with an estrogen such as ethinyl estradiol can thus be used for the production of a liquid contraceptive composition, the systemic bioavailability of which, upon spraying onto oral mucosal membranes, is very high (and possibly almost complete) when compared with the prodrug desogestrel that is orally ingested. This systemic bioavailability is obtained without requiring a percutaneous absorption promoter or a matrix former.

### SUMMARY OF THE INVENTION

The subject invention provides a liquid oral contraceptive composition formulated for transmucosal administration.

### FIGURES

FIG. 1: schematic description of the manufacturing process of an oral contraceptive spray of the subject invention.

FIG. 2: Etonogestrel serum concentration (on a linear scale) versus time profiles after (i) an orally ingested Marvelon® tablet (0.150 mg desogestrel, 0.030 mg ethinyl estradiol) and (ii) a liquid containing 0.157 mg of etonogestrel and 0.030 mg of ethinyl estradiol administered to the oral mucosa.

FIG. 3: Ethinyl estradiol plasma concentration (on a linear scale) versus time profiles after (i) an orally ingested Marvelon® tablet and (ii) a liquid containing 0.157 mg of etonogestrel and 0.030 mg of ethinyl estradiol administered to the oral mucosa.

FIG. 4: Etonogestrel serum concentration (on a logarithmic scale) versus time profiles after (i) an orally ingested Marvelon® tablet and (ii) a liquid containing 0.157 mg of etonogestrel and 0.030 mg of ethinyl estradiol administered to the oral mucosa.

FIG. 5: Ethinyl estradiol plasma concentration (on a logarithmic scale) versus time profiles after (i) an orally ingested Marvelon® tablet and (ii) a liquid containing 0.157 mg of etonogestrel and 0.030 mg of ethinyl estradiol administered to the oral mucosa.

FIG. 6: Etonogestrel serum concentration (on a linear scale) versus time profiles after (i) an orally ingested Cerazette® tablet and (ii) a liquid containing 0.075 mg of etonogestrel administered to the oral mucosa.

FIG. 7: Etonogestrel serum concentration (on a logarithmic scale) versus time profiles after (i) an orally ingested Cerazette® tablet and (ii) a liquid containing 0.075 mg of etonogestrel administered to the oral mucosa.

### DETAILED DESCRIPTION

The subject invention provides a liquid oral contraceptive composition formulated for mucosal administration.

The subject liquid oral contraceptive composition can be used in any oral contraceptive regimen, i.e. monophasic, biphasic, triphasic etc.

The subject liquid composition is typically administered once a day to a mucosal surface. In one embodiment, it is administered to the oral mucosal surface. In a specific embodiment it is administered sublingually, supralingually or buccally.

The term “spray” as used herein encompasses a spray, a liquid, droplets, a mist, a fluid flow or any other liquid form.

The term “spraying” as used herein encompasses spraying of a spray, a mist, droplets, a fluid flow or any other liquid form for oral mucosal administration.

The liquid composition of the invention can be administered by any suitable device capable of generating a spray, a mist, droplets, a fluid flow or any other liquid form for oral mucosal administration such as a metered dose container, a pump container, a spray device, a fluid dispenser, a device generating a fluid flow, a fluid distributor pump, a liquid distribution device, a liquid droplet spray device, a droplet generator and the like.

The device administers the composition of the invention in the form of a spray, a mist, droplets, a fluid flow or any other liquid form for oral mucosal administration.

A device as used herein means any such device with a sufficient amount of the composition of the subject invention for daily administration for at least 7 days. In a specific embodiment, the device contains a sufficient amount of the composition of the subject invention for daily administration for at least 21 days. In another embodiment, the device contains a sufficient amount of the composition of the subject invention for daily administration for at least 24 days. In yet another embodiment, the device contains a sufficient amount of the composition of the subject invention for daily administration for at least a month (i.e. 28, 29, 30 or 31 days). In yet another embodiment, the device contains a sufficient amount of the composition of the subject invention for daily administration for at least 63 days. In a specific embodiment, the device contains a sufficient amount of the composition of the subject invention for daily administration for at least 84 days. In a specific embodiment, the device contains a sufficient amount of the composition of the subject invention for daily administration for at least 90 days.

The formulation of the subject invention may comprise a progestogen, an estrogen or combinations thereof. It may optionally also contain other active ingredients such as anti-microbials, folic acid, vitamins etc.

Progestogen as used in the subject invention is etonogestrel or desogestrel or 3-β-hydroxy desogestrel.

Estrogen as used in the subject invention is ethinyl-estradiol or estradiol or an ester thereof or a (pseudo)polymorph thereof.

In a specific embodiment of the subject invention, the progestogen is etonogestrel. In another embodiment, the progestogen is desogestrel or 3-β-hydroxydesogestrel.

In one embodiment of the subject invention the estrogen is ethinyl estradiol. In another embodiment, the estrogen is estradiol or an ester thereof or a (pseudo)polymorph thereof, such as estradiol hemihydrate.

In a specific embodiment, the progestogen is etonogestrel and the estrogen is ethinyl estradiol.

In one embodiment, administration of the contraceptive formulation of the subject invention is continuous, i.e. daily, i.e. every day without steroid-free days. This embodiment, which is known as a continuous regimen, is particularly useful when the contraceptive formulation of the subject invention contains only progestogen (such as etonogestrel).

In another embodiment, particularly useful when the contraceptive formulation of the subject invention comprises both a progestogen (such as etonogestrel) and an estrogen (such as ethinyl estradiol), administration of the contraceptive formulation of the subject invention is on the basis of a regular 21-7 or 24-4 contraceptive regimen as are known in the art, e.g. the composition is administered daily for 21 days, whereafter there is a hormone-free 7 day period. Thereafter, administration of the composition of the subject invention is resumed. Alternatively, the composition is administered daily for 24 days, whereafter there is a hormone-free 4 day period whereafter administration of the composition of the subject invention is resumed.

In yet another embodiment, administration of the contraceptive formulation of the subject invention is on the basis of any one of the calendar regimens as described in WO/US2006/23382, which is hereby incorporated by reference.

The subject invention thus inter alia provides a liquid contraceptive formulation comprising etonogestrel provided that the formulation does not contain a matrix former or a percutaneous absorption promoter, which is a hydroxy acid or a salt thereof.

In one embodiment, this formulation comprises a contraceptively effective daily dose of etonogestrel between 0.04 mg and 0.08 mg or between 0.04 and 0.075 mg. In a specific embodiment, this formulation comprises a contraceptively effective daily dose of etonogestrel between 0.045 mg and 0.08 mg or between 0.045 and 0.075 mg. Alternatively, the effective daily dose of etonogestrel lies between 0.05 and 0.08 mg or between 0.05 and 0.075 mg. In another alternative embodiment, the effective daily dose of etonogestrel lies between 0.04 and 0.065 mg or between 0.045 and 0.065 mg.

It is further envisaged that a liquid contraceptive formulation of the subject invention may, in addition to etonogestrel, further comprise ethinyl estradiol. In such an embodiment, the formulation comprises a contraceptively effective daily dose of etonogestrel between 0.08 and 0.16 mg and of ethinyl estradiol between 0.015 and 0.03 mg. In a specific embodiment, this formulation comprises a contraceptively effective daily dose of etonogestrel between 0.09 and 0.145 mg and of ethinyl estradiol between 0.02 and 0.03 mg.

In a particular embodiment of the subject invention, the liquid formulation is a spray formulation.

In one embodiment, the formulation of the subject invention is used for female contraception. Such a method of female contraception comprises administering to an oral mucosal surface of a female a contraceptively effective metered spray dose of a formulation of the subject invention. In a specific embodiment, the contraceptively effective metered spray dose is administered continuously daily. In another embodiment, the contraceptively effective metered spray dose is administered daily for at least 21 days.

The subject invention further provides a use of a metered dose of an oral mucosal spray comprising etonogestrel for daily administration for female contraception. In one embodiment, the daily contraceptively effective metered dose comprises between 0.04 mg and 0.08 mg etonogestrel. In a specific embodiment, the daily contraceptively effective metered dose comprises between 0.04 mg and 0.075 mg etonogestrel, in particular between 0.045 and 0.08 or between 0.045 and 0.075 mg. Alternatively, the effective metered dose comprises between 0.05 and 0.08 mg or between 0.05 and 0.075 mg of etonogestrel. In another alternative embodiment, the effective metered dose comprises between 0.04 and 0.065 mg or between 0.045 and 0.065 mg of etonogestrel.

The subject invention further provides a use of a metered dose of an oral mucosal spray comprising etonogestrel and ethinyl estradiol for daily administration for female contraception. In such an embodiment, the daily contraceptively effective metered dose comprises between 0.08 and 0.16 mg etonogestrel and between 0.015 and 0.03 mg ethinyl estradiol. In a specific embodiment, the daily contraceptively effective metered dose comprises between 0.09 and 0.145 mg etonogestrel and between 0.02 and 0.03 mg ethinyl estradiol.

The subject invention further provides a contraceptive kit comprising a device comprising a liquid composition for oral mucosal administration by spraying, said composition comprising a contraceptively effective amount of etonogestrel for at least 7 daily metered doses. In a specific embodiment, the composition in such a kit further comprises ethinyl estradiol.

The subject invention also provides a contraceptive kit comprising a device comprising a liquid composition for oral mucosal administration by spraying, said composition comprising a contraceptively effective amount of etonogestrel for at least 21 daily metered doses. In a specific embodiment, the composition in such a kit further comprises ethinyl estradiol.

The subject invention also provides a contraceptive kit comprising a device comprising a liquid composition for oral mucosal administration by spraying, said composition comprising a contraceptively effective amount of etonogestrel for at least 28, 29, 30 or 31 daily metered doses. In a specific embodiment, the composition in such a kit further comprises ethinyl estradiol.

The subject invention further provides a drug delivery system for female contraception comprising a device comprising a liquid composition for oral mucosal administration by spraying comprising at least 7 metered daily doses each comprising between 0.04 mg and 0.08 mg of etonogestrel. In a specific embodiment, each metered dose comprises between 0.045 and 0.08 mg or between 0.045 and 0.075 mg of etonogestrel. Alternatively, each metered dose comprises between 0.05 and 0.08 mg or between 0.05 and 0.075 mg of etonogestrel. In another alternative embodiment, each metered dose comprises between 0.04 and 0.065 mg or between 0.045 and 0.065 mg of etonogestrel.

The subject invention further provides a drug delivery system for female contraception comprising a device comprising a liquid composition for oral mucosal administration by spraying comprising at least 7 metered daily doses each comprising between 0.08 and 0.16 mg etonogestrel and between 0.015 and 0.03 mg of ethinyl estradiol. In a specific embodiment, each metered dose comprises between 0.09 and 0.145 mg etonogestrel and between 0.02 and 0.03 mg of ethinyl estradiol.

The present invention is further described in the following examples, which are not in any way intended to limit the scope of the invention as claimed.

### EXAMPLES

### Example 1

An example of a formulation for oral mucosal administration of etonogestrel, optionally including ethinyl estradiol, is depicted in Tables 1 and 2.

Initially, an “oral spray bulk solution” is made by mixing the ingredients as indicated in Table 1. The oral spray is then prepared by addition of the active compounds as depicted in Table 2.

TABLE 1

| | Quantity per |
|---|---|
| Component | mL |
| Propylene glycol | 25% (v/v) |
| Ethanol | 25% (v/v) |
| Glycerol | 25% (v/v) |
| Water | 25% (v/v) |
| Peppermint oil* | 0.83 mg |

*Contribution to total volume is negligible due to its small amount

TABLE 2

| | Quantity per | | Quantity per | |
|---|---|---|---|---|
| Component | unit | | mL | |
| Etonogestrel | 0.157* | mg | 1.570 | mg |
| Ethinyl estradiol | 0.030 | mg | 0.300 | mg |
| Bulk solution | To 0.1 | mL | To 1 | mL |

*equivalent on a molar basis to a dose of 0.150 mg desogestrel

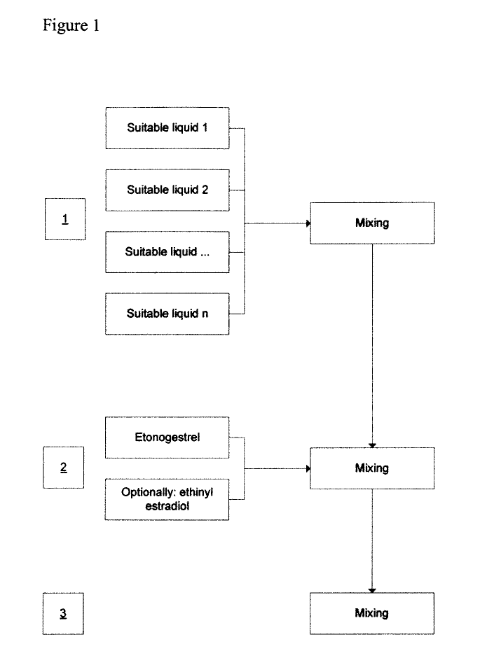

FIG. 1 schematically depicts the manufacturing process of the production of the oral spray.

A sufficient amount to administer 0.1 mL of the solution so prepared was filled into a 0.2 mL glass vial. The glass vial was placed in a device to enable spraying the solution in the mouth.

### Example 2

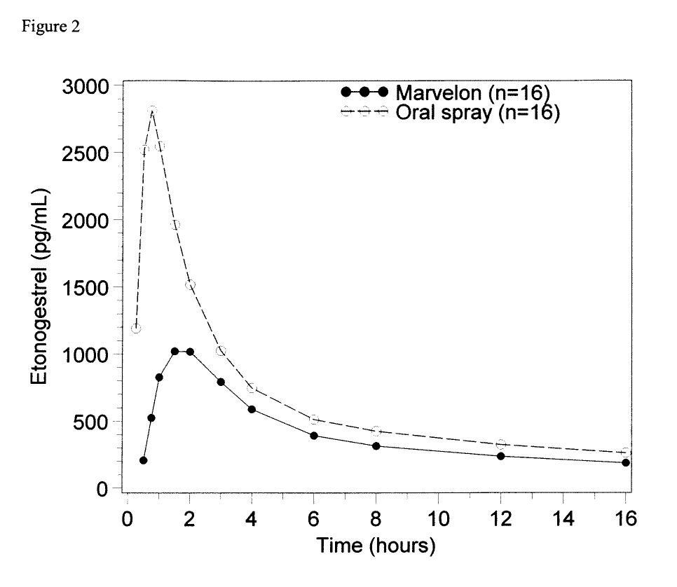

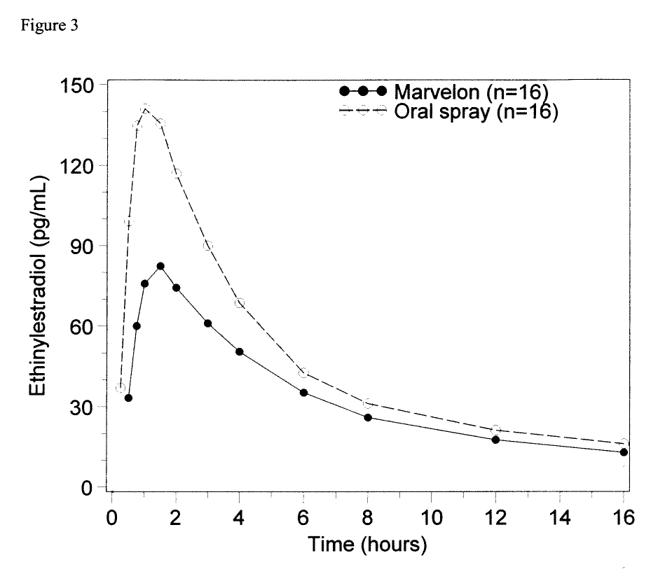

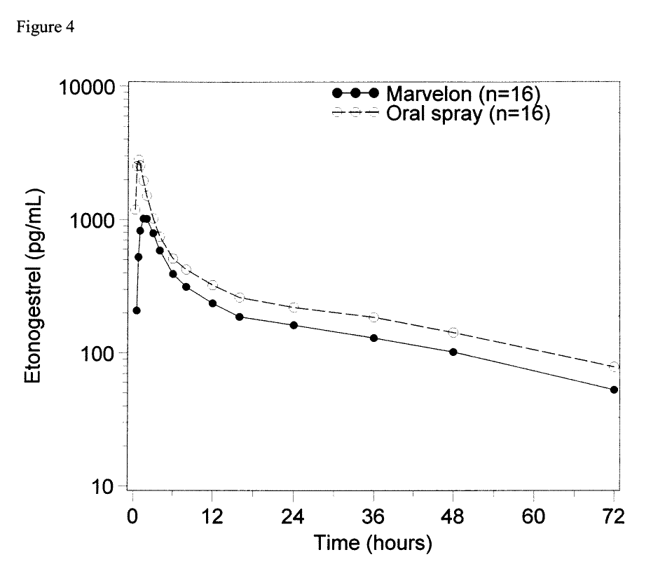

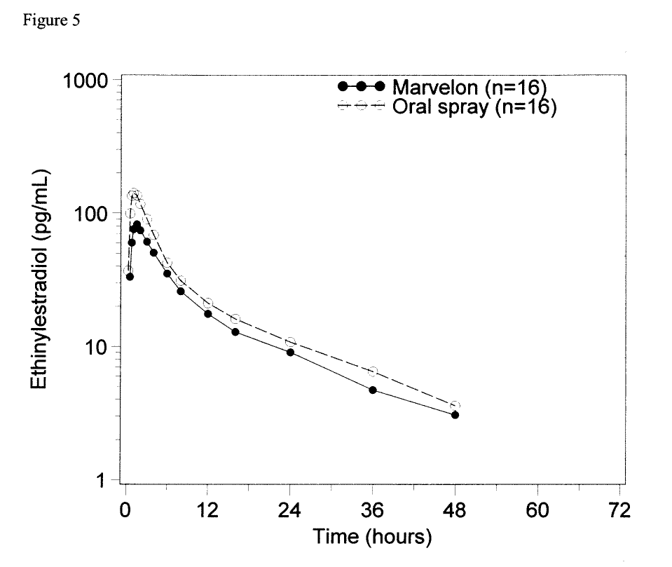

Sixteen healthy female subjects were treated with 0.1 ml of the oral spray as described in Example 1 or with Marvelon® tablets (150 μg desogestrel, 30 μg ethinyl estradiol) in a cross over design. The spray was administered sublingually. Marvelon® was ingested. Serum concentration versus time profiles were obtained for etonogestrel and plasma concentration versus time profiles were obtained for ethinyl estradiol. FIGS. 2, 3, 4 and 5 show the average values of the serum/plasma concentrations versus time profiles.

FIGS. 2, 3, 4 and 5 show that oral mucosal administration of both etonogestrel and ethinyl estradiol leads to much higher levels of etonogestrel (in serum) and ethinyl estradiol (in plasma) when compared to oral ingestion of equivalent amounts of desogestrel and ethinyl estradiol using a tablet.

Table 3 summarizes key-characteristics of the curves. It shows that the effect of exposure as expressed by Area Under the Curve (either AUC₀₋ₜₗₐₛₜ or AUC_{0-χ}) is significant, the effect on maximum plasma concentration (Cₘₐₓ) is even higher. This leads to the conclusion that a lower dose of either etonogestrel, or ethinyl estradiol, or both is possible for oral mucosal administration.

TABLE 3

| | | Geometric mean | |
|---|---|---|---|
| Analyte | Parameter | Spray | Marvelon^{R} |
| Etonogestrel | Cₘₐₓ (pg/mL) | 3023 | 1342 |
| | AUC₀₋ₜₗₐₛₜ (pg * h/mL) | 20130 | 13092 |
| | AUC_{0-∞} (pg * h/mL) | 25532 | 17015 |
| | tₘₐₓ (h)* | 0.76 | 1.50 |
| Ethinyl estradiol | Cₘₐₓ (pg/mL) | 150 | 85 |
| | AUC₀₋ₜₗₐₛₜ (pg * h/mL) | 1047 | 746 |
| | AUC_{0-∞} (pg * h/mL) | 1140 | 833 |
| | tₘₐₓ (h)* | 1.00 | 1.50 |

*Presented mean refers to arithmetic mean

### Example 3

Solutions were prepared essentially according to the procedure as described in Example 1. The solubility of each one of the active ingredients desogestrel, 3-β-hydroxydesogestrel, 3-keto desogestrel and ethinyl estradiol in these solutions was tested. The results are listed in Table 4 and 5, in which Table 4 depicts the composition of the liquids and Table 5 depicts the solubilities of each one of the compounds indicated in these liquid solutions.

It is practically difficult to administer less than about 0.05 ml volume in an accurate manner. In addition, a volume of above 0.5 ml may result in a swallowing reflex which could cause swallowing (ingestion) of the liquid composition rather than absorption through the oral mucosal membranes. Therefore, in order to assure absorption through the oral mucosal membranes, the volume of liquid to be administered is between 0.05 to 0.5 ml and in a specific embodiment, between 0.05 to 0.3 mL.

The contraceptive target dose of desogestrel, 3-β-hydroxy desogestrel or etonogestrel is maximally about 0.160 mg and that of ethinyl estradiol is maximally 0.030 mg. This implies that the solubility of the desogestrel, 3-β-hydroxy desogestrel or etonogestrel should preferably be at least 0.32 to 3.2 mg/ml (i.e. 0.032% to 0.32%) and that of ethinyl estradiol between 0.6 and 0.06 mg/mL (i.e. 0.006% to 0.06%). The results thus show that it is possible to administer the compounds in a true solution, without matrix formers and the like, and it is not necessary to increase solubility by complex formation such as micelles.

TABLE 4

| | | | | Propylene | Polysorbate |
|---|---|---|---|---|---|
| | Water | Ethanol | Glycerol | Glycol | 80 |
| Liquid 1 | 25 | 20 | 25 | 25 | 5 |
| Liquid 2 | 25 | 25 | 20 | 25 | 5 |
| Liquid 3 | 25 | 30 | 15 | 25 | 5 |
| Liquid 4 | 25 | 25 | 25 | 25 | 0 |
| Liquid 5 | 30 | 30 | 25 | 15 | 0 |

Values are percentages by volume (v/v)

TABLE 5

| | Liquid 1 | Liquid 2 | Liquid 3 | Liquid 4 | Liquid 5 |
|---|---|---|---|---|---|
| Desogestrel | 11.3 | 12.4 | 13.4 | 2.1 | 1.5 |
| Etonogestrel | 5.5 | 5.8 | 7.3 | 4.2 | 3.4 |
| 3-β-Hydroxy-desogestrel | ≧8.8 | ≧7.0 | ≧6.1 | ≧6.9 | ≧8.7 |
| Ethinyl Estradiol | ≧10.9 | ≧10.8 | ≧14.1 | 11.2 | 12.4 |

Values in mg/mL, ≧ means “larger than”.

### Example 4

Sixteen healthy female subjects were treated with 0.1 ml of oral spray comprising the liquid as depicted in table 6 to administer 75 μg etonogestrel, or with Cerazette® tablets (75 μg desogestrel) in a cross over design. The spray has been administered sublingually. Cerazette® has been ingested. Serum concentration versus time profiles were obtained for etonogestrel.

TABLE 6

| | Quantity per |
|---|---|
| Component | mL |
| Propylene glycol | 15% (v/v) |
| Ethanol | 30% (v/v) |
| Glycerol | 25% (v/v) |
| Water | 30% (v/v) |
| Peppermint oil* | 0.83 mg |
| Etonogestrel** | 0.75 mg |

*Contribution to total volume is negligible due to its small amount
**amount not corrected to be equal on a molar basis with desogestrel for reason of the very small correction factor

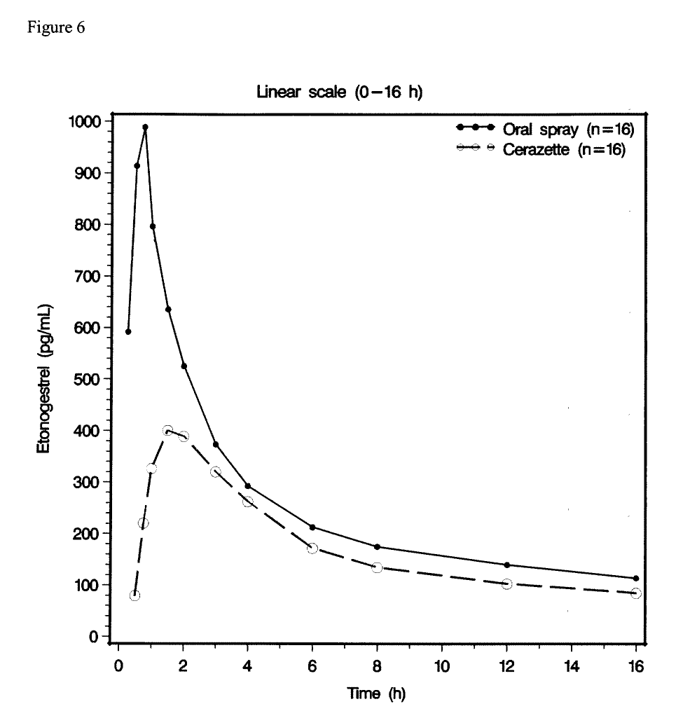

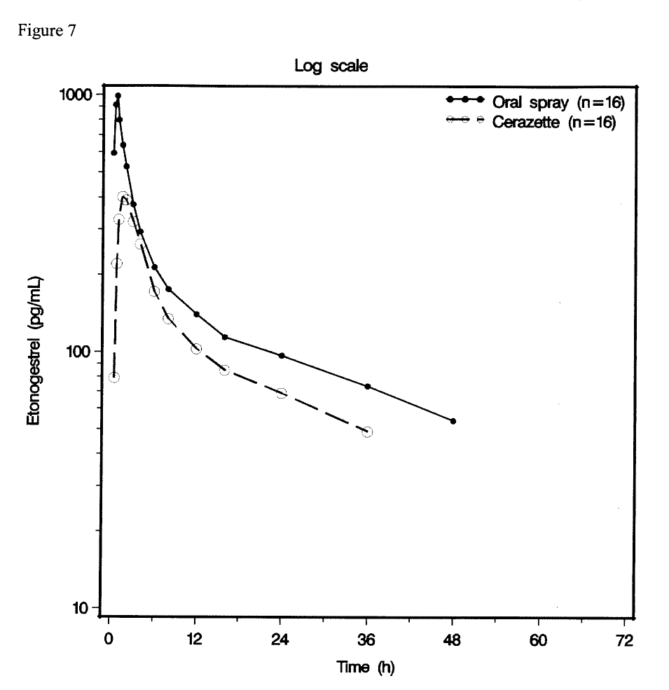

FIGS. 6 and 7 show that oral mucosal administration of etonogestrel leads to much higher levels of etonogestrel (in serum) when compared to oral ingestion of an equivalent amount of desogestrel using a tablet. FIG. 6 shows the concentration versus time profile with a linear concentration-axis, FIG. 7 shows the same results, but on a log-linear scale

Table 7 summarizes key-characteristics of the serum concentration versus time curves. It shows that the effect of exposure as expressed by Area Under the Curve (either AUC₀₋ₜₗₐₛₜ or AUC_{0-χ}) is significant, the effect on maximum plasma concentration (Cₘₐₓ) is even higher. This leads to the conclusion that a lower dose of either etonogestrel is possible for oral mucosal administration to achieve a similar pharmacological effect.

TABLE 7

Geometric mean

| | Oral | |
|---|---|---|
| Parameter (unit) | spray | Cerazette ® |
| Cₘₐₓ (pg/mL) | 1092 | 581 |
| AUC₀₋ₜₗₐₛₜ (ng · h/mL) | 7.86 | 5.03 |
| AUC_{0-inf} (ng · h/mL) | 11.0 | 7.09 |
| tₘₐₓ (h)* | 0.75 | 1.50 |

*Presented mean refers to arithmetic mean
Source: Study 293002

What is claimed:

1. A liquid contraceptive formulation for oral mucosal administration comprising etonogestrel provided that the formulation does not contain a matrix former or a percutaneous absorption promoter which is a hydroxy acid or a salt thereof.

2. The formulation of claim 1, further comprising ethinyl estradiol.

3. The formulation of claim 1, wherein the contraceptively effective daily dose of etonogestrel is between 0.04 mg and 0.08 mg.

4. The formulation of claim 3, wherein the contraceptively effective daily dose of etonogestrel is between 0.04 and 0.075 mg.

5. The formulation of claim 3, wherein the contraceptively effective daily dose of etonogestrel is between 0.045 and 0.08 mg.

6. The formulation of claim 3, wherein the contraceptively effective daily dose of etonogestrel is between 0.045 mg and 0.075 mg.

7. The formulation of claim 2, wherein the contraceptively effective daily dose of etonogestrel is between 0.08 and 0.16 mg and of ethinyl estradiol is between 0.015 and 0.03 mg.

8. The formulation of claim 7, wherein the contraceptively effective daily dose of etonogestrel is between 0.09 and 0.145 mg and of ethinyl estradiol is between 0.02 and 0.03 mg.

9. The formulation of claim 1, wherein the liquid formulation is a spray formulation.

10. A method of female contraception comprising administering to an oral mucosal surface of a female a contraceptively effective metered spray dose of a formulation of claim 1.

11. The method of claim 10, wherein the contraceptively effective metered spray dose is administered daily in a continuous regimen.

12. The method of claim 10, wherein the contraceptively effective metered spray dose is administered daily for at least 21 days.

13. The method of claim 10, wherein the spray dose is administered sublingually.

14. A contraceptive kit comprising a device comprising a liquid composition for oral mucosal administration by spraying, said composition comprising a contraceptively effective amount of etonogestrel for at least 7 daily metered doses.

15. The contraceptive kit according to claim 14, wherein the composition comprises a contraceptively effective amount of etonogestrel for at least 21 daily metered doses.

16. The contraceptive kit of claim 14, wherein the composition comprises a contraceptively effective amount of etonogestrel for at least 28, 29, 30 or 31 daily metered doses.

17. The contraceptive kit of claim 14, wherein the composition further comprises ethinyl estradiol.

18. A drug delivery system for female contraception comprising a device comprising a liquid composition for oral mucosal administration by spraying comprising at least 7 metered daily doses each comprising between 0.04 mg and 0.08 mg of etonogestrel.

19. The drug delivery system of claim 18, wherein each metered dose comprises between 0.045 and 0.08 mg of etonogestrel.

20. The drug delivery system of claim 18, wherein each metered dose comprises between 0.04 and 0.075 mg of etonogestrel.

21. The drug delivery system of claim 18, wherein each metered dose comprises between 0.045 and 0.075 mg of etonogestrel.

22. A drug delivery system for female contraception comprising a device comprising a liquid composition for oral mucosal administration by spraying comprising at least 7 metered daily doses each comprising between 0.08 and 0.16 mg etonogestrel and between 0.015 and 0.03 mg of ethinyl estradiol.

23. The drug delivery system of claim 22, wherein each metered dose comprises between 0.09 and 0.145 mg etonogestrel and between 0.02 and 0.03 mg of ethinyl estradiol.
